(19) United States
(12) Patent Application Publication
(10) Pub. No.: US 2011/0023153 A1
(43) Pub. Date: Jan. 27, 2011

(54) GENOMIC EDITING OF GENES INVOLVED IN ALZHEIMER'S DISEASE

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,993

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/00; A01K 67/00; C12N 5/10; C12N 9/16; C07H 21/04

(52) U.S. Cl. 800/3; 800/13; 800/15; 800/16; 800/17; 800/14; 435/325; 435/350; 435/352; 435/366; 435/363; 435/351; 435/353; 435/196; 536/23.2

(57) ABSTRACT

The present invention provides genetically modified animals and cells comprising edited chromosomal sequences encoding proteins associated with AD. In particular, the animals or cells are generated using a zinc finger nuclease-mediated editing process. Also provided are methods of using the genetically modified animals or cells disclosed herein to study AD development and methods of assessing the effects of agents in genetically modified animals and cells comprising edited chromosomal sequences encoding proteins associated with AD.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding proteins associated with Alzheimer's disease. In particular, the invention relates to the use of zinc finger nuclease-mediated processes to edit chromosomal sequences encoding proteins associated with Alzheimer's disease.

### BACKGROUND OF THE INVENTION

Alzheimer's disease (AD) is the most common form of progressive dementia in aged humans, and it is genetically heterogeneous. Currently existing animal models do not recapitulate major hallmarks of the disease as it is observed in humans. The available animal models comprising mutant genes encoding proteins associated with AD also produce highly variable phenotypes, making translations to human disease and therapy development problematic. For example, the behavioral performance of mouse models of AD mutations tested for learning and memory can be difficult to interpret, and thus can be a poor indicator of responses in humans. In addition, baseline intelligence in mouse strains varies, resulting in unpredictable behavioral traits in crossbred animals with different genetic backgrounds where multiple mutations may be combined. What are needed are animal models with AD-related proteins genetically modified to provide research tools that allow the elucidation of mechanisms underlying development and progression of AD.

### SUMMARY OF THE INVENTION

One aspect of the present disclosure encompasses a genetically modified animal comprising at least one edited chromosomal sequence encoding a protein associated with AD.

A further aspect provides a non-human embryo comprising at least one RNA molecule encoding a zinc finger nuclease that recognizes a chromosomal sequence encoding a protein associated with AD, and, optionally, at least one donor polynucleotide comprising a sequence encoding a protein associated with AD.

Another aspect provides a genetically modified cell comprising at least one edited chromosomal sequence encoding a protein associated with AD.

An alternate aspect provides a zinc finger nuclease comprising (a) a zinc finger DNA binding domain that binds a sequence having at least about 80% sequence identity with a sequence chosen from SEQ ID NO:7, 8, 9, 10, 11, and 12; and (b) a cleavage domain.

Another aspect provides a nucleic acid sequence that is bound by a zinc finger nuclease. The nucleic acid sequence has at least about 80% sequence identity with a sequence chosen from SEQ ID NO:7, 8, 9, 10, 11, and 12.

An additional aspect encompasses a method for assessing the effect of genetically modified protein associated with AD on the progression or symptoms of AD or an AD-related disorder in an animal. The method comprises comparing a wild type animal to a genetically modified animal comprising at least one edited chromosomal sequence encoding a protein associated with AD, and measuring an AD-related phenotype.

Yet another aspect encompasses a method for assessing the effect of an agent on the progression or symptoms of AD. The method comprises contacting a first genetically modified animal comprising at least one edited chromosomal sequence encoding a protein associated with AD with the agent and measuring an AD-related phenotype in the first animal. The method further comprises comparing results of the AD-related phenotype in the first animal to results obtained from a second genetically modified animal comprising the same edited chromosomal sequence encoding a protein associated with AD, wherein the second animal is not contacted with the agent.

Other aspects and features of the disclosure are described more thoroughly below.

### REFERENCE TO COLOR FIGURES

The application file contains at least one figure executed in color. Copies of this patent application publication with color figure will be provided by the Office upon request and payment of the necessary fee.

### BRIEF DESCRIPTION OF THE FIGURES

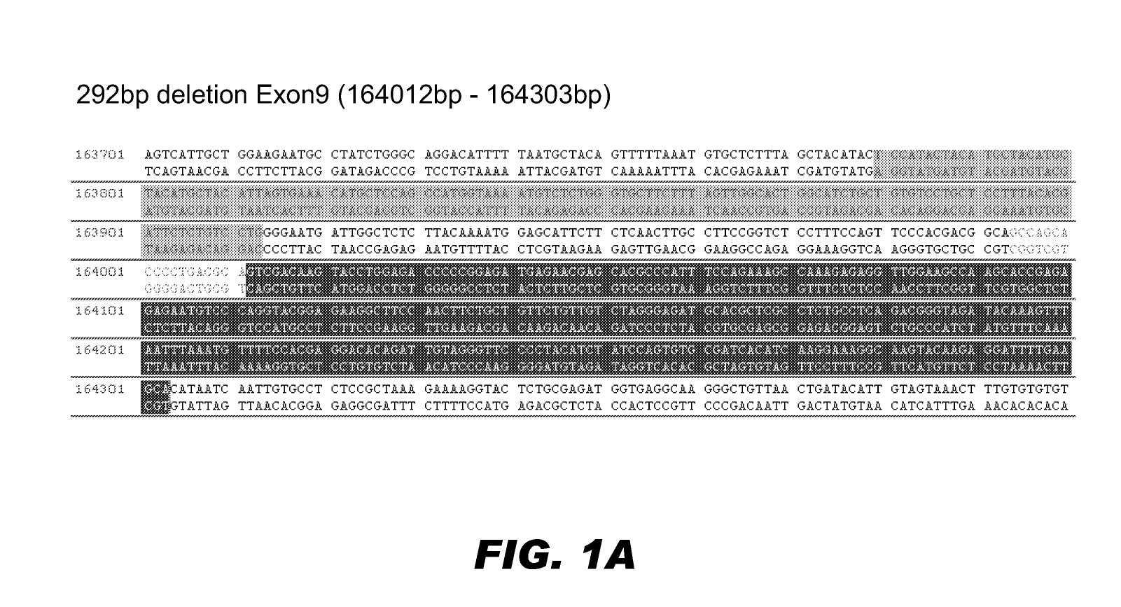

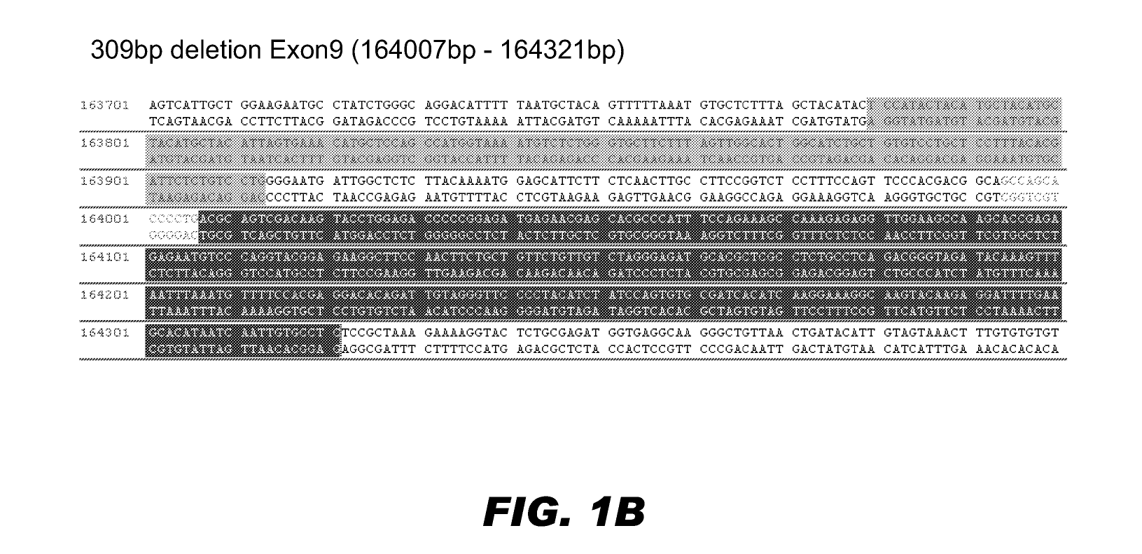

FIG. 1 presents the DNA sequences of edited APP loci in two animals. (A) Shows a region of the rat APP locus (SEQ ID NO:1) in which 292 bp is deleted from exon 9. (B) Presents a region of the rat APP locus (SEQ ID NO:2) in which there is a 309 bp deletion in exon 9. The exon is shown in green; the target site is presented in yellow, and the deletion is shown in dark blue.

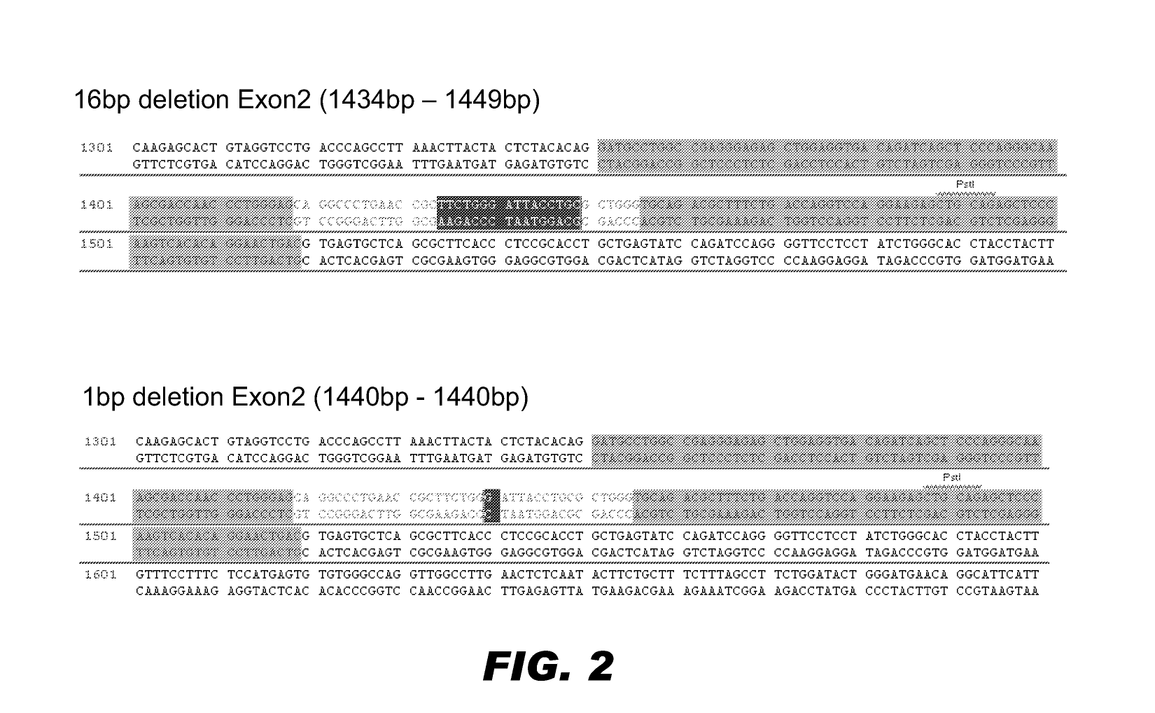

FIG. 2 presents the DNA sequences of two edited ApoE loci. The upper sequence (SEQ ID NO:3) has a 16 bp deletion in the target sequence of exon 2, and the lower sequence (SEQ ID NO:4) has a 1 bp deletion in the target sequence of exon 2. The exon sequence is shown in green; the target site is presented in yellow, and the deletions are shown in dark blue.

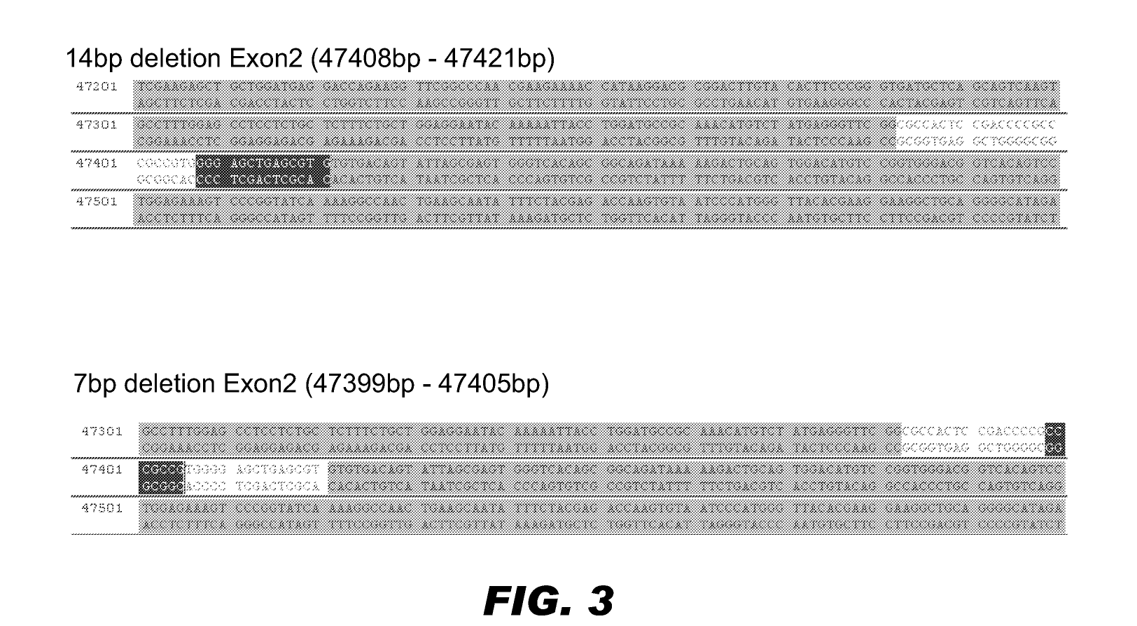

FIG. 3 shows the DNA sequences of edited BDNF loci in two animals. The upper sequence (SEQ ID NO:5) has a 14 bp deletion in the target sequence in exon 2, and the lower sequence (SEQ ID NO:6) has a 7 bp deletion in the target sequence in exon 2. The exon is shown in green; the target site is presented in yellow, and the deletions are shown in dark blue.

### DETAILED DESCRIPTION OF THE INVENTION

The present disclosure provides a genetically modified animal or animal cell comprising at least one edited chromosomal sequence encoding a protein associated with AD. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock-out” or a “conditional knock-out.” Similarly, a genetically modified animal comprising an integrated sequence may be termed a “knock-in” or a “conditional knock-in.” As detailed below, a knock-in animal may be a humanized animal. Furthermore, a genetically modified animal comprising a modified chromosomal sequence may comprise a targeted point mutation(s) or other modification such that an altered protein product is produced. The chromosomal sequence encoding the protein associated with AD generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with AD using targeted zinc finger nuclease technology is rapid, precise, and highly efficient.

### (I) Genetically Modified Animals.

One aspect of the present disclosure provides a genetically modified animal in which at least one chromosomal sequence encoding a protein associated with AD has been edited. For example, the edited chromosomal sequence may be inactivated such that the sequence is not transcribed and/or a functional protein associated with AD is not produced. Alternatively, the chromosomal sequence may be edited such that the sequence is over-expressed and a functional protein associated with AD is over-produced. The edited chromosomal sequence may also be modified such that it codes for an altered protein associated with AD. For example, the chromosomal sequence may be modified such that at least one nucleotide is changed and the expressed protein associated with AD comprises at least one changed amino acid residue (i.e., a missense mutation). The chromosomal sequence may be modified to comprise more than one missense mutation such that more than one amino acid is changed. Additionally, the chromosomal sequence may be modified to have a three nucleotide deletion or insertion such that the expressed protein associated with AD comprises a single amino acid deletion or insertion, provided such a protein is functional. The modified protein associated with AD may have altered substrate specificity, altered enzyme activity, altered kinetic rates, and so forth. Furthermore, the edited chromosomal sequence encoding a protein associated with AD may comprise a sequence encoding a protein associated with AD integrated into the genome of the animal. The chromosomally integrated sequence may encode an endogenous protein associated with AD normally found in the animal, or the integrated sequence may encode an exogenous orthologous protein associated with AD, or combinations of both. The genetically modified animal disclosed herein may be heterozygous for the edited chromosomal sequence encoding a protein associated with AD. Alternatively, the genetically modified animal may be homozygous for the edited chromosomal sequence encoding a protein associated with AD.

In one embodiment, the genetically modified animal may comprise at least one inactivated chromosomal sequence encoding a protein associated with AD. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional protein associated with AD is not produced. The inactivated chromosomal sequence comprises no exogenously introduced sequence. Such an animal may be termed a “knock-out.” Also included herein are genetically modified animals in which two, three, or more chromosomal sequences encoding proteins associated with AD are inactivated.

In another embodiment, the genetically modified animal may comprise at least one edited chromosomal sequence encoding a protein associated with AD such that the sequence is over-expressed and a functional protein associated with AD is over-produced. For example, the regulatory regions controlling the expression of the protein associated with AD may be altered such that the protein associated with AD is over-expressed.

In yet another embodiment, the genetically modified animal may comprise at least one chromosomally integrated sequence encoding a protein associated with AD. For example, an exogenous sequence encoding an orthologous or an endogenous protein associated with AD may be integrated into a chromosomal sequence encoding a protein associated with AD such that the chromosomal sequence is inactivated, but wherein the exogenous sequence encoding the orthologous or endogenous protein associated with AD may be expressed or overexpressed. In such a case, the sequence encoding the orthologous or endogenous protein associated with AD may be operably linked to a promoter control sequence. Alternatively, an exogenous sequence encoding an orthologous or endogenous protein associated with AD may be integrated into a chromosomal sequence without affecting expression of a chromosomal sequence. For example, an exogenous sequence encoding a protein associated with AD may be integrated into a “safe harbor” locus, such as the Rosa26 locus, HPRT locus, or AAV locus, wherein the exogenous sequence encoding the orthologous or endogenous protein associated with AD may be expressed or overexpressed. An animal comprising a chromosomally integrated sequence encoding a protein associated with AD may be called a “knock-in,” and it should be understood that in such an iteration of the animal, no selectable marker is present. The present disclosure also encompasses genetically modified animals in which 2, 3, 4, 5, 6, 7, 8, 9, 10, 11, 12, 13, 14, 15, or more sequences encoding proteins associated with AD are integrated into the genome.

The chromosomally integrated sequence encoding a protein associated with AD may encode the wild-type form of the protein associated with AD. Alternatively, the chromosomally integrated sequence encoding a protein associated with AD may comprise at least one modification such that an altered version of the protein associated with AD is produced. In some embodiments, the chromosomally integrated sequence encoding a protein associated with AD comprises at least one modification such that the altered version of the protein associated with AD produced causes AD. In other embodiments, the chromosomally integrated sequence encoding a protein associated with AD comprises at least one modification such that the altered version of the protein associated with AD protects against AD.

In an additional embodiment, the genetically modified animal may be a “humanized” animal comprising at least one chromosomally integrated sequence encoding a functional human protein associated with AD. The functional human AD-associated protein may have no corresponding ortholog in the genetically modified animal. Alternatively, the wild-type animal from which the genetically modified animal is derived may comprise an ortholog corresponding to the functional protein associated with AD. In this case, the orthologous sequence in the “humanized” animal is inactivated such that no functional protein is made and the “humanized” animal comprises at least one chromosomally integrated sequence encoding the human protein associated with AD. For example, a humanized animal may comprise an inactivated endogenous APP sequence and a chromosomally integrated human APP sequence. Those of skill in the art appreciate that “humanized” animals may be generated by crossing a knock-out animal with a knock-in animal comprising the chromosomally integrated sequence.

In yet another embodiment, the genetically modified animal may comprise at least one edited chromosomal sequence encoding a protein associated with AD such that the expression pattern of the protein is altered. For example, regulatory regions controlling the expression of the protein, such as a promoter or transcription binding site, may be altered such that the addiction-related protein is over-produced, or the tissue-specific or temporal expression of the protein is altered, or a combination thereof. Alternatively, the expression pattern of the protein associated with AD may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a protein associated with AD. The genetically modified animal comprising the lox-flanked chromosomal sequence encoding a protein associated with AD may then be crossed with another genetically modified animal expressing Cre recombinase. Progeny animals comprising the lox-flanked chromosomal sequence encoding a protein associated with AD and the Cre recombinase are then produced, and the lox-flanked chromosomal sequence encoding a protein associated with AD is recombined, leading to deletion or inversion of the chromosomal sequence encoding the protein. Expression of Cre recombinase may be temporally and conditionally regulated to effect temporally and conditionally regulated recombination of the chromosomal sequence encoding a protein associated with AD.

(a) Proteins Associated with AD

The present disclosure comprises editing of any chromosomal sequences that encode proteins associated with AD. The AD-related proteins are typically selected based on an experimental association of the AD-related protein to an AD disorder. For example, the production rate or circulating concentration of an AD-related protein may be elevated or depressed in a population having an AD disorder relative to a population lacking the AD disorder. Differences in protein levels may be assessed using proteomic techniques including but not limited to Western blot, immunohistochemical staining, enzyme linked immunosorbent assay (ELISA), and mass spectrometry. Alternatively, the AD-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

By way of non-limiting example, proteins associated with AD include but are not limited to the proteins listed in Table A.

TABLE A

| Chromosomal | |
|---|---|
| Sequence | Encoded Protein |
| ALAS2 | Delta-aminolevulinate synthase 2 (ALAS2) |
| ABCA1 | ATP-binding cassette transporter (ABCA1) |
| ACE | Angiotensin I-converting enzyme (ACE) |
| APOE | Apolipoprotein E precursor (APOE) |
| APP | amyloid precursor protein (APP) |
| AQP1 | aquaporin 1 protein (AQP1) |
| BIN1 | Myc box-dependent-interacting protein 1 or |
| | bridging integrator 1 protein (BIN1) |
| BDNF | brain-derived neurotrophic factor (BDNF) |
| BTNL8 | Butyrophilin-like protein 8 (BTNL8) |
| C1ORF49 | chromosome 1 open reading frame 49 |
| CDH4 | Cadherin-4 |
| CHRNB2 | Neuronal acetylcholine receptor subunit beta-2 |
| CKLFSF2 | CKLF-like MARVEL transmembrane domain- |
| | containing protein 2 (CKLFSF2) |
| CLEC4E | C-type lectin domain family 4, member e (CLEC4E) |
| CLU | clusterin protein (also known as |
| | apoplipoprotein J) |
| CR1 | Erythrocyte complement receptor 1 (CR1, also |
| | known as CD35, C3b/C4b receptor and immune |
| | adherence receptor) |
| CR1L | Erythrocyte complement receptor 1 (CR1L) |
| CSF3R | granulocyte colony-stimulating factor 3 receptor |
| | (CSF3R) |
| CST3 | Cystatin C or cystatin 3 |
| CYP2C | Cytochrome P450 2C |
| DAPK1 | Death-associated protein kinase 1 (DAPK1) |
| ESR1 | Estrogen receptor 1 |
| FCAR | Fc fragment of IgA receptor (FCAR, also known |
| | as CD89) |
| FCGR3B | Fc fragment of IgG, low affinity IIIb, receptor |
| | (FCGR3B or CD16b) |
| FFA2 | Free fatty acid receptor 2 (FFA2) |
| FGA | Fibrinogen (Factor I) |
| GAB2 | GRB2-associated-binding protein 2 (GAB2) |
| GAB2 | GRB2-associated-binding protein 2 (GAB2) |
| GALP | Galanin-like peptide |
| GAPDHS | Glyceraldehyde-3-phosphate dehydrogenase, |
| | spermatogenic (GAPDHS) |
| GMPB | GMBP |
| HP | Haptoglobin (HP) |
| HTR7 | 5-hydroxytryptamine (serotonin) receptor 7 |
| | (adenylate cyclase-coupled) |
| IDE | Insulin degrading enzyme |
| IF127 | IF127 |
| IFI6 | Interferon, alpha-inducible protein 6 (IFI6) |
| IFIT2 | Interferon-induced protein with |
| | tetratricopeptide repeats 2 (IFIT2) |
| IL1RN | interleukin-1 receptor antagonist (IL-1RA) |
| IL8RA | Interleukin 8 receptor, alpha (IL8RA or CD181) |
| IL8RB | Interleukin 8 receptor, beta (IL8RB) |
| JAG1 | Jagged 1 (JAG1) |
| KCNJ15 | Potassium inwardly-rectifying channel, subfamily |
| | J, member 15 (KCNJ15) |
| LRP6 | Low-density lipoprotein receptor-related |
| | protein 6 (LRP6) |
| MAPT | microtubule-associated protein tau (MAPT) |
| MARK4 | MAP/microtubule affinity-regulating |
| | kinase 4 (MARK4) |
| MPHOSPH1 | M-phase phosphoprotein 1 |
| MTHFR | 5,10-methylenetetrahydrofolate reductase |
| MX2 | Interferon-induced GTP-binding protein Mx2 |
| NBN | Nibrin, also known as NBN |
| NCSTN | Nicastrin |
| NIACR2 | Niacin receptor 2 (NIACR2, also known as |
| | GPR109B) |
| NMNAT3 | nicotinamide nucleotide adenylyltransferase 3 |
| NTM | Neurotrimin |
| (or HNT) | |
| ORM1 | Orosmucoid 1 (ORM1) or Alpha-1-acid |
| | glycoprotein 1 |
| P2RY13 | P2Y purinoceptor 13 (P2RY13) |
| PBEF1 | Nicotinamide phosphoribosyltransferase |
| | (NAmPRTase or Nampt) also known as pre-B-cell |
| | colony-enhancing factor 1 (PBEF1) or visfatin |
| PCK1 | Phosphoenolpyruvate carboxykinase |
| PICALM | phosphatidylinositol binding clathrin assembly |
| | protein (PICALM) |
| PLAU | Urokinase-type plasminogen activator (PLAU) |
| PLXNC1 | Plexin C1 (PLXNC1) |
| PRNP | Prion protein |
| PSEN1 | presenilin 1 protein (PSEN1) |
| PSEN2 | presenilin 2 protein (PSEN2) |
| PTPRA | protein tyrosine phosphatase receptor type A |
| | protein (PTPRA) |
| RALGPS2 | Ral GEF with PH domain and SH3 binding motif 2 |
| | (RALGPS2) |
| RGSL2 | regulator of G-protein signaling like 2 (RGSL2) |
| SELENBP1 | Selenium binding protein 1 (SELNBP1) |
| SLC25A37 | Mitoferrin-1 |
| SORL1 | sortilin-related receptor L(DLR class) A |
| | repeats-containing protein (SORL1) |
| TF | Transferrin |
| TFAM | Mitochondrial transcription factor A |
| TNF | Tumor necrosis factor |
| TNFRSF10C | Tumor necrosis factor receptor superfamily |
| | member 10C (TNFRSF10C) |
| TNFSF10 | Tumor necrosis factor receptor superfamily, |
| (TRAIL) | member 10a (TNFSF10) |
| UBA1 | ubiquitin-like modifier activating enzyme 1 |
| | (UBA1) |
| UBA3 | NEDD8-activating enzyme E1 catalytic subunit |
| | protein (UBE1C) |
| UBB | ubiquitin B protein (UBB) |
| UBQLN1 | Ubiquilin-1 |
| UCHL1 | ubiquitin carboxyl-terminal esterase L1 protein |
| | (UCHL1) |
| UCHL3 | ubiquitin carboxyl-terminal hydrolase isozyme L3 |
| | protein (UCHL3) |
| VLDLR | very low density lipoprotein receptor protein |
| | (VLDLR) |

In exemplary embodiments, the proteins associated with AD whose chromosomal sequence is edited may be the very low density lipoprotein receptor protein (VLDLR) encoded by the VLDLR gene, the ubiquitin-like modifier activating enzyme 1 (UBA1) encoded by the UBA1 gene, the NEDD8-activating enzyme E1 catalytic subunit protein (UBE1C) encoded by the UBA3 gene, the aquaporin 1 protein (AQP1) encoded by the AQP1 gene, the ubiquitin carboxyl-terminal esterase L1 protein (UCHL1) encoded by the UCHL1 gene, the ubiquitin carboxyl-terminal hydrolase isozyme L3 protein (UCHL3) encoded by the UCHL3 gene, the ubiquitin B protein (UBB) encoded by the UBB gene, the microtubule-associated protein tau (MAPT) encoded by the MAPT gene, the protein tyrosine phosphatase receptor type A protein (PTPRA) encoded by the PTPRA gene, the phosphatidylinositol binding clathrin assembly protein (PICALM) encoded by the PICALM gene, the clusterin protein (also known as apoplipoprotein J) encoded by the CLU gene, the presenilin 1 protein encoded by the PSEN1 gene, the presenilin 2 protein encoded by the PSEN2 gene, the sortilin-related receptor L(DLR class) A repeats-containing protein (SORL1) protein encoded by the SORL1 gene, the amyloid precursor protein (APP) encoded by the APP gene, the Apolipoprotein E precursor (APOE) encoded by the APOE gene, or the brain-derived neurotrophic factor (BDNF) encoded by the BDNF gene. In an exemplary embodiment, the genetically modified animal is a rat, and the edited chromosomal sequence encoding the protein associated with AD is as listed in Table B.

TABLE B

| Chromosomal | | NCBI Reference |
|---|---|---|
| Sequence | Encoded Protein | Sequence |
| APP | amyloid precursor protein (APP) | NM_019288 |
| AQP1 | aquaporin 1 protein (AQP1) | NM_012778 |
| BDNF | Brain-derived neurotrophic factor | NM_012513 |
| CLU | clusterin protein (also known as | NM_053021 |
| | apoplipoprotein J) | |
| MAPT | microtubule-associated protein | NM_017212 |
| | tau (MAPT) | |
| PICALM | phosphatidylinositol binding | NM_053554 |
| | clathrin assembly protein (PICALM) | |
| PSEN1 | presenilin 1 protein (PSEN1) | NM_019163 |
| PSEN2 | presenilin 2 protein (PSEN2) | NM_031087 |
| PTPRA | protein tyrosine phosphatase | NM_012763 |
| | receptor type A protein (PTPRA) | |
| SORL1 | sortilin-related receptor L(DLR | NM_053519, |
| | class) A repeats-containing | XM_001065506, |
| | protein (SORL1) | XM_217115 |
| UBA1 | ubiquitin-like modifier activating | NM_001014080 |
| | enzyme 1 (UBA1) | |
| UBA3 | NEDD8-activating enzyme E1 | NM_057205 |
| | catalytic subunit protein (UBE1C) | |
| UBB | ubiquitin B protein (UBB) | NM_138895 |
| UCHL1 | ubiquitin carboxyl-terminal | NM_017237 |
| | esterase L1 protein (UCHL1) | |
| UCHL3 | ubiquitin carboxyl-terminal | NM_001110165 |
| | hydrolase isozyme L3 | |
| | protein (UCHL3) | |
| VLDLR | very low density lipoprotein | NM_013155 |
| | receptor protein (VLDLR) | |

The animal or cell may comprise 1, 2, 3, 4, 5, 6, 7, 8, 9,10, 11, 12, 13, 14, 15 or more disrupted chromosomal sequences encoding a protein associated with AD and zero, 1, 2, 3, 4, 5, 6, 7, 8, 9, 10, 11, 12, 13, 14, 15 or more chromosomally integrated sequences encoding a protein associated with AD.

The edited or integrated chromosomal sequence may be modified to encode an altered protein associated with AD. A number of mutations in AD-related chromosomal sequences have been associated with AD. For instance, the V7171 (i.e. valine at position 717 is changed to isoleucine) missense mutation in APP causes familial AD. Multiple mutations in the presenilin-1 protein, such as H163R (i.e. histidine at position 163 is changed to arginine), A246E (i.e. alanine at position 246 is changed to glutamate), L286V (i.e. leucine at position 286 is changed to valine) and C410Y (i.e. cysteine at position 410 is changed to tyrosine) cause familial Alzheimer's type 3. Mutations in the presenilin-2 protein, such as N141 I (i.e. asparagine at position 141 is changed to isoleucine), M239V (i.e. methionine at position 239 is changed to valine), and D439A (i.e. aspartate at position 439 is changed to alanine) cause familial Alzheimer's type 4. Other associations of genetic variants in AD-associated genes and disease are known in the art. See, for example, Waring et al. (2008) Arch. Neurol. 65:329-334, the disclosure of which is incorporated by reference herein in its entirety.

### (b) Animals

The term “animal,” as used herein, refers to a non-human animal. Moreover, the animal does not include exogenously introduced transposon sequences. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of commonly used rat strains suitable for genetic manipulation include Dahl Salt-Sensitive, Fischer 344, Lewis, Long Evans Hooded, Sprague-Dawley and Wistar. In another iteration of the invention, the animal does not comprise a genetically modified mouse. In each of the foregoing iterations of suitable animals for the invention, the animal does not include exogenously introduced, randomly integrated transposon sequences.

(c) Proteins Associated with AD

The protein associated with AD may be from any of the animals listed above. Furthermore, the protein associated with AD may be a human protein associated with AD. Additionally, the protein associated with AD may be a bacterial, fungal, or plant protein. The type of animal and the source of the protein can and will vary. As an example, the genetically modified animal may be a rat, cat, dog, or pig, and the protein associated with AD may be human. Alternatively, the genetically modified animal may be a rat, cat, or pig, and the protein associated with AD may be canine. One of skill in the art will readily appreciate that numerous combinations are possible and are encompassed by the present invention. In an exemplary embodiment, the genetically modified animal is a rat, and the protein associated with AD is human.

Additionally, the sequence encoding the protein associated with AD may be modified to include a tag or reporter gene as is well-known. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence encoding a protein associated with AD. The genetically modified cell or cell line may be derived from any of the genetically modified animals disclosed herein. Alternatively, the chromosomal sequence coding a protein associated with AD may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genomic Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

An exemplary zinc finger DNA binding domain recognizes and binds a sequence having at least about 80% sequence identity with a sequence chosen from SEQ ID NO:7, 8, 9, 10, 11, and 12. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fokl cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:1538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences encoding proteins associated with AD may further comprise introducing at least one donor polynucleotide comprising a sequence encoding a protein associated with AD into the embryo or cell. A donor polynucleotide comprises at least three components: the sequence coding the protein associated with AD, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding the protein associated with AD may be a BAC.

The sequence of the donor polynucleotide that encodes the protein associated with AD may include coding (i.e., exon) sequence, as well as intron sequences and upstream regulatory sequences (such as, e.g., a promoter). Depending upon the identity and the source of the sequence encoding the protein associated with AD, the size of the sequence encoding the protein associated with AD will vary. For example, the sequence encoding the protein associated with AD may range in size from about 1 kb to about 5,000 kb.

The donor polynucleotide also comprises upstream and downstream sequence flanking the sequence encoding the protein associated with AD. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the protein associated with AD, a double stranded break introduced into the chromosomal sequence by the zinc finger nuclease is repaired, via homologous recombination with the donor polynucleotide, such that the sequence encoding the protein associated with AD is integrated into the chromosome. The presence of a double-stranded break facilitates integration of the sequence encoding the protein associated with AD. A donor polynucleotide may be physically integrated or, alternatively, the donor polynucleotide may be used as a template for repair of the break, resulting in the introduction of the sequence encoding the protein associated with AD as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences encoding a protein associated with AD may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85; 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially.

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the protein associated with AD in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated APP chromosomal sequence may be crossed with animal B comprising a chromosomally integrated sequence encoding a human APP to give rise to a “humanized” APP offspring comprising both the inactivated APP chromosomal sequence and the chromosomally integrated human APP gene. Similarly, an animal comprising an inactivated PSEN1 chromosomal sequence may be crossed with an animal comprising a chromosomally integrated sequence encoding the human PSEN1 protein to generate “humanized” PSEN1 offspring. Moreover, a humanized APP animal may be crossed with a humanized PSEN1 animal to create a humanized APP/PSEN1 animal. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild type genetic backgrounds, genetic backgrounds with deletion mutations, genetic backgrounds with another targeted integration, and genetic backgrounds with non-targeted integrations.

### (IV) Applications

A further aspect of the present disclosure encompasses a method for using the genetically modified animals. In one embodiment, the genetically modified animals may be used to study the effects of targeted mutations on the development and/or progression of AD using measures commonly used in the study of AD. Commonly used measures in the study of AD include without limit, learning and memory, anxiety, depression, addiction, and sensory-motor functions, as well as functional, pathological, metabolic, or biochemical assays. Those of skill in the art are familiar with other suitable measures or indicators of AD. In general, such measures may be made in comparison to wild type littermates.

Other measures of behavior may include assessments of spontaneous behavior. Spontaneous behavior may be assessed using any one or more methods of spontaneous behavioral observations known in the art. In general, any spontaneous behavior within a known behavioral repertoire of an animal may be observed, including movement, posture, social interaction, rearing, sleeping, blinking, eating, drinking, urinating, defecating, mating, and aggression. An extensive battery of observations for quantifying the spontaneous behavior of mice and rats is well-known in the art, including but not limited to home-cage observations such as body position, respiration, tonic involuntary movement, unusual motor behavior such as pacing or rocking, catatonic behavior, vocalization, palpebral closure, mating frequency, running wheel behavior, nest building, and frequency of aggressive interactions.

Behavioral testing may be used to assess other indications of the development and/or progression of AD, including but not limited to reflex function, motor function, long-term memory function, anxiety, and depression. Performance during behavioral testing may be assessed using any number of behavioral tests known in the art. The particular type of performance test may depend upon at least one of several factors including the behavioral repertoire of the animal and the purpose of the testing. For example, non-limiting examples of tests for assessing the reflex function of rats include assessments of approach response, touch response, eyelid reflex, pinna reflex, sound response, tail pinch response, pupillary reflex, and righting reflex. Non-limiting examples of behavioral tests suitable for assessing the motor function of rats includes open field locomoter activity assessment, the rotarod test, the grip strength test, the cylinder test, the limb-placement or grid walk test, the vertical pole test, the Inverted grid test, the adhesive removal test, the painted paw or catwalk (gait) tests, the beam traversal test, and the inclined plane test. Non-limiting examples of behavioral tests suitable for assessing the long-term memory function of rats include the elevated plus maze test, the Morris water maze swim test, contextual fear conditioning, the Y-maze test, the T-maze test, the novel object recognition test, the active avoidance test, the passive (inhibitory) avoidance test, the radial arm maze test, the two-choice swim test, the hole board test, the olfactory discrimination (go-no-go) test, and the pre-pulse inhibition test. Non-limiting examples of behavioral tests suitable for assessing the anxiety of rats include the open field locomotion assessment, observations of marble-burying behavior, the elevated plus maze test, the light/dark box test. Non-limiting examples of behavioral tests suitable for assessing the depression of rats includes the forced swim test, the tail suspension test, the hot plate test, the tail suspension test, anhedonia observations, and the novelty suppressed feeding test.

In another embodiment, the animals of the invention may be used to study the effects of the mutations on the progression of a disease state or disorder other than AD, but which is also associated with AD-related proteins, using measures commonly used in the study of said disease state or disorder. Non limiting examples of disease states or disorders other than AD that may be associated with AD-related proteins include dementia, congenital cerebellar ataxia, mental retardation such as learning and memory defects, lissencephaly, tauopathy or fibrilization, amyloidosis, neurodegeneration, Parkinsonism, progressive supranuclear palsy, Pick disease, male infertility, prostate and breast cancer, squamous cell carcinoma, lymphoma, leukemia, and atherosclerosis.

In another embodiment, the genetically modified animals and cells may be used for assessing the effect(s) of an agent on AD. Alternatively, the animals and cells of the invention may be used for assessing the effect(s) of an agent on the progression of a disease state or disorder other than AD, but which is also associated with AD-related proteins. Suitable agents include without limit pharmaceutically active ingredients, AD drugs, suitable biologics, and other therapeutic agents. For example, the effect(s) of an agent may be measured in a “humanized” genetically modified rat, such that the information gained therefrom may be used to predict the effect of the agent in a human. In general, the method comprises contacting a genetically modified animal comprising at least one edited chromosomal sequence encoding a protein associated with AD with the agent, and comparing results of a selected parameter to results obtained from contacting a control animal with the same agent.

Selected parameters include but are not limited to (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); and (i) extrahepatic contribution to metabolic rate and clearance of the agent or its metabolite(s); and j) the ability of the agent to reduce the incidence or indications of addiction, or to reduce the pathology resulting from the introduction of at least one chromosomal sequence encoding a protein associated with AD into the genome of a genetically-modified animal.

For example, an ADME-Tox profile of therapeutic compounds or combinations of therapeutic agents may be assessed using a genetically modified animal comprising at least one edited chromosomal sequence encoding a protein associated with AD. The ADME-Tox profile may include assessments of at least one or more physiologic and metabolic consequences of administering the therapeutic compound or combination of therapeutic agents. In addition, the ADME-Tox profile may assess behavioral effects such as addiction, depression, or sensorimotor function in response to the therapeutic compound or combination of therapeutic agents.

Also provided are methods to assess the effect(s) of an agent in an isolated cell comprising at least one edited chromosomal sequence encoding a protein associated with AD, as well as methods of using lysates of such cells (or cells derived from a genetically modified animal disclosed herein) to assess the effect(s) of an agent. For example, the role of a particular protein associated with AD in the metabolism of a particular agent may be determined using such methods. Similarly, substrate specificity and pharmacokinetic parameter may be readily determined using such methods.

Yet another aspect encompasses a method for assessing the efficacy of a potential gene therapy strategy. That is, a chromosomal sequence encoding a protein associated with AD may be modified such that the genetically modified animal may have an altered response to the development and/or progression of AD as compared to a non treated animal. Stated another way, a mutated gene that predisposes an animal to AD may be “corrected” through gene therapy.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or exchange molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### Examples

The following examples are included to illustrate the invention.

### Example 1

### Genome Editing of APP Locus

Zinc finger nucleases (ZFNs) that target and cleave the APP locus of rats were designed, assembled, and validated using strategies and procedures previously described (see Geurts et al. Science (2009) 325:433). ZFN design made use of an archive of pre-validated 1-finger and 2-finger modules. The rat APP gene region was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay identified a pair of active ZFNs that edited the APP locus. The zinc finger binding sites were 5′-GCCAGCACCCCTGACgcag-3′ (SEQ ID NO:7) and 5′-tcGACAAGTACCTGGAG-3′ (SEQ ID NO:8).

To mediate editing of the APP gene locus in animals, fertilized rat embryos were microinjected with mRNA encoding the active pair of ZFNs using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the APP locus was PCR amplified using appropriate primers. The amplified DNA was subcloned into a suitable vector and sequenced using standard methods. FIG. 1 presents edited APP loci in two founder animals; one had a 292 bp deletion in exon 9 (FIG. 1A) and the other had a 309 bp deletion in exon 9 (FIG. 1B).

### Example 2

### Genome Editing of ApoE Locus

ZFNs with activity at the ApoE locus were identified as described above. That is, the rat ApoE gene (NM_{—}138828) was scanned for putative zinc finger binding sites, and pairs of ZFNs were assembled and tested essentially as described in Example 1. It was found that the ZFN pair targeted to bind 5′-aaGCGGTTCAGGGCCTGctcccagggtt-3′ (SEQ ID NO:9; contact sites in upper case) and 5′-ggGATTACCTGcGCTGGGtgcagacgct-3′ (SEQ ID NO:10) cleaved the ApoE locus.

Fertilized one-cell embryos were injected with mRNAs encoding the active ZFN pair as described above in Example 1. The resultant animals were analyzed as detailed in Example 1. FIG. 2 presents two edited ApoE loci. One animal had a 16 bp deletion in the target sequence of exon 2, and a second animal had a 1 bp deletion in the target sequence of exon 2. These deletions disrupt the reading frame of the ApoE coding region.

### Example 3

### Genome Editing of BDNF Locus

To identify ZFNs that target and cleave the BDNF locus, the rat BDNF gene (NM_{—}012513) was scanned for putative zinc finger binding sites. The ZFNs pairs were assembled and tested essentially as described in Example 1. This analysis revealed that the ZFN pair targeted to bind 5′-cgGGGTCGGAGtGGCGCCgaaccctcat-3′ (SEQ ID NO:11) and 5′-cgGGGTCGGAGtGGCGCCgaaccctcat-3′ (SEQ ID NO:12) edited the BDNF locus.

Fertilized rat embryos were microinjected with mRNAs encoding the active ZNF pair and analyzed essentially as described above in Example 1. FIG. 3 presents edited BDNF loci in two founder animals; one had a 14 bp deletion in the target sequence in exon 2 and the other had a 7 bp deletion in the target sequence in exon 2.

The genetically modified rats were observed for phenotypic changes. Homozygous animals died within 2 weeks of birth. Heterozygous and homozygous animals were smaller in size than corresponding control animals (i.e., derived from embryos microinjected with GFP mRNA).

### Example 4

### Genome Editing of PSEN1 in a Model Organism

ZFN-mediated genome editing may be used to study the effects of a “knockout” mutation in an AD-related chromosomal sequence, such as a chromosomal sequence encoding the PSEN1 protein, in a genetically modified model animal and cells derived from the animal. Such a model animal may be a rat. In general, ZFNs that bind to the rat chromosomal sequence encoding the PSEN1 protein associated with AD may be used to introduce a deletion or insertion such that the coding region of the PSEN1 gene is disrupted such that a functional PSEN1 protein may not be produced.

Suitable fertilized embryos may be microinjected with capped, polyadenylated mRNA encoding the ZFN essentially as detailed above in Example 1. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the PSEN1 “knockout” may be assessed in the genetically modified rat or progeny thereof. Furthermore, molecular analyses of AD-related pathways may be performed in cells derived from the genetically modified animal comprising a PSEN1 “knockout”.

### Example 5

### Generation of a Humanized Rat Expressing a Mutant Form of Human PSEN2

Missense mutations in PSEN2, a part of the enzymatic complex that cleaves amyloid beta peptide from APP, cause type 4 familial AD. One such mutation is the M239V missense mutation where the methionine residue acid at position 239 in PSEN2 is replaced with a valine residue. ZFN-mediated genome editing may be used to generate a humanized rat wherein the rat PSEN2 gene is replaced with a mutant form of the human PSEN2 gene comprising the M239V mutation. Such a humanized rat may be used to study the development of the diseases associated with the mutant human PSEN2 protein. In addition, the humanized rat may be used to assess the efficacy of potential therapeutic agents targeted at the pathway leading to AD comprising PSEN2.

The genetically modified rat may be generated using the methods described in the Examples above. However, to generate the humanized rat, the ZFN mRNA may be co-injected with the human chromosomal sequence encoding the mutant PSEN2 protein into the rat embryo. The rat chromosomal sequence may then be replaced by the mutant human sequence by homologous recombination, and a humanized rat expressing a mutant form of the PSEN2 protein may be produced.

The table below presents the amino acid sequences of helices of the active ZFNs.

| Name | Sequence of Zinc Finger Helices | SEQ ID NO: |
|---|---|---|
| ApoE | RSDALSV DSSHRTR RSDNLSE TSGSLTR | 13 |
| | RSDDLTR | |
| ApoE | RSDHLSR QSSDLRR RSDVLSA DRSNRIK | 14 |
| | TSSNLSR | |
| BDNF | DRSDLSR DRSHLAR RSHNLAR RSDDLSK | 15 |
| | RSAHLSR | |
| BDNF | RSDNLAR QSSDLRR RSSHLSR RSDALSR | 16 |
| | DRSDLSR | |

What is claimed is:

1. A genetically modified animal comprising at least one edited chromosomal sequence encoding a protein associated with AD.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no functional protein is produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a protein associated with AD.

6. The genetically modified animal of claim 1, wherein the edited chromosomal sequence is modified such that the protein associated with AD is over-produced.

7. The genetically modified animal of claim 1, further comprising a conditional knock-out system for conditional expression of the protein associated with AD.

8. The genetically modified animal of claim 1, wherein the edited chromosomal sequence comprises an integrated reporter sequence

9. The genetically modified animal of claim 1, wherein the protein associated with AD is chosen from the proteins listed in Table A, and combinations thereof.

10. The genetically modified animal of claim 1, wherein the protein associated with AD is chosen from APP, AQP1, clusterin, MAPT, PICALM, PSEN1, PSEN2, PTPRA, SORL1, UBA1, UBA3, UBB, UCHL1, UCHL3, VLDLR, ApoE, BDNF, and combinations thereof.

11. The genetically modified animal of claim 1, wherein the animal is heterozygous or homozygous for the at least one edited chromosomal sequence.

12. The genetically modified animal of claim 1, wherein the animal is an embryo, a juvenile, or an adult.

13. The genetically modified animal of claim 1, wherein the animal is chosen from bovine, canine, equine, feline, ovine, porcine, non-human primate, and rodent.

14. The genetically modified animal of claim 5, wherein the animal is rat and the chromosomally integrated sequence encoding a protein associated with AD is human.

15. A non-human embryo, the embryo comprising at least one RNA molecule encoding a zinc finger nuclease that recognizes a chromosomal sequence encoding a protein associated with AD, and, optionally, at least one donor polynucleotide comprising a sequence encoding a protein associated with AD.

16. The non-human embryo of claim 15, wherein the protein associated with AD is chosen from APP, AQP1, clusterin, MAPT, PICALM, PSEN1, PSEN2, PTPRA, SORL1, UBA1, UBA3, UBB, UCHL1, UCHL3, VLDLR, ApoE, and BDNF, and combinations thereof.

17. The non-human embryo of claim 15, wherein the embryo is chosen from bovine, canine, equine, feline, ovine, porcine, non-human primate, and rodent.

18. The non-human embryo of claim 15, wherein the embryo is rat and the donor polynucleotide comprising a sequence encoding a protein associated with AD is human.

19. A genetically modified cell, the cell comprising at least one edited chromosomal sequence encoding a protein associated with AD.

20. The genetically modified cell of claim 19, wherein the edited chromosomal sequence is inactivated, modified, or comprises an integrated sequence.

21. The genetically modified cell of claim 19, wherein the edited chromosomal sequence is inactivated such that no functional protein is produced.

22. The genetically modified cell of claim 21, wherein the inactivated chromosomal sequence comprises no exogenously introduced sequence.

23. The genetically modified cell of claim 21, further comprising at least one chromosomally integrated sequence encoding a protein associated with AD.

24. The genetically modified cell of claim 19, wherein the edited chromosomal sequence is modified such that the protein associated with AD is over-produced.

25. The genetically modified cell of claim 19, further comprising a conditional knock-out system for conditional expression of the protein associated with AD.

26. The genetically modified cell of claim 19, wherein the edited chromosomal sequence comprises an integrated reporter sequence

27. The genetically modified cell of claim 19, wherein the protein associated with AD is chosen from the proteins listed in Table A, and combinations thereof.

28. The genetically modified cell of either claim 19, wherein the protein associated with AD is chosen from APP, AQP1, clusterin, MAPT, PICALM, PSEN1, PSEN2, PTPRA, SORL1, UBA1, UBA3, UBB, UCHL1, UCHL3, VLDLR, ApoE, BDNF, and combinations thereof.

29. The genetically modified cell of claim 19, wherein the cell is heterozygous or homozygous for the at least one edited chromosomal sequence.

30. The genetically modified cell of claim 19, wherein the cell is of bovine, canine, equine, feline, human, ovine, porcine, non-human primate, or rodent origin.

31. The genetically modified cell of claim 23, wherein the cell is of rat origin and the chromosomally integrated sequence encoding a protein associated with AD is human.

32. A zinc finger nuclease, the zinc finger nuclease comprising:
a) a zinc finger DNA binding domain that binds a sequence having at least about 80% sequence identity with a sequence chosen from SEQ ID NO:7, 8, 9, 10, 11, and 12; and
b) a cleavage domain.

33. The zinc finger nuclease of claim 32, wherein the sequence identity is at least about 85%, 90%, 95%, or 100%.

34. The zinc finger nuclease of claim 32, wherein the DNA binding domain comprises at least three finger recognition regions.

35. The zinc finger nuclease of claim 32, wherein the cleavage domain is a wild-type or an engineered Fokl cleavage domain.

36. A nucleic acid sequence bound by a zinc finger nuclease, the nucleic acid sequence having at least about 80% sequence identity with a sequence chosen from SEQ ID NO:7, 8, 9, 10, 11, and 12.

37. The nucleic acid sequence of claim 36, wherein the sequence identity is at least about 85%, 90%, 95%, or 100%.

38. A method for assessing the effect of a genetically modified protein associated with AD on the progression or symptoms of AD or an AD-related disorder in an animal, the method comprising comparing a wild type animal to a genetically modified animal comprising at least one edited chromosomal sequence encoding a protein associated with AD, and measuring an AD-related phenotype.

39. The method of claim 38, wherein the at least one edited chromosomal sequence is inactivated such that no functional protein is produced.

40. The method of claim 38, wherein the at least one edited chromosomal sequence is inactivated such that the protein associated with AD is over-produced.

41. The method of claim 39, further comprising at least one chromosomally integrated sequence encoding a protein associated with AD.

42. The method of claim 38, wherein the protein associated with AD is chosen from the proteins listed in Table A, and combinations thereof.

43. The method of claim 38, wherein the protein associated with AD is chosen from APP, AQP1, clusterin, MAPT, PICALM, PSEN1, PSEN2, PTPRA, SORL1, UBA1, UBA3, UBB, UCHL1, UCHL3, VLDLR, ApoE, BDNF, and combinations thereof.

44. The method of claim 38, wherein the AD-related disorder is chosen from dementia, congenital cerebellar ataxia, mental retardation such as learning and memory defects, lissencephaly, tauopathy or fibrilization, amyloidosis, neurodegeneration, Parkinsonism, progressive supranuclear palsy, Pick disease, male infertility, prostate and breast cancer, squamous cell carcinoma, lymphoma, leukemia, and atherosclerosis.

45. A method for assessing the effect of an agent on progression or symptoms of AD, the method comprising:
a) contacting a first genetically modified animal comprising at least one edited chromosomal sequence encoding a protein associated with AD with an agent;
b) measuring an AD-related phenotype in the first genetically modified animal, and
c) comparing results of the AD-related phenotype in (b) to results obtained from a second genetically modified animal comprising the same edited chromosomal sequence as the first genetically modified animal, wherein the second genetically modified animal is not contacted with the agent.

46. The method of claim 45, wherein the agent is a pharmaceutically active ingredient, a biologically active agent, a therapeutic agent, or an AD drug.

47. The method of claim 45, wherein the at least one edited chromosomal sequence is inactivated such that no functional protein is produced.

48. The method of claim 47, further comprising at least one chromosomally integrated sequence encoding a protein associated with AD.

49. The method of claim 45, wherein the protein associated with AD is chosen from the proteins listed in Table A, and combinations thereof.

50. The method of claim 45, wherein the protein associated with AD is chosen from APP, AQP1, clusterin, MAPT, PICALM, PSEN1, PSEN2, PTPRA, SORL1, UBA1, UBA3, UBB, UCHL1, UCHL3, VLDLR, ApoE, BDNF, and combinations thereof.


SEQUENCE LISTING (16 sequences)

SEQ ID NO: 1 - DNA, 700 bp, Rattus rattus
agtcattgctggaagaatgcctatctgggcaggacatttttaatgctacagtttttaaatgtgctctttagctacatactccatactacatgctacatgctacatgctacattagtgaaacatgctccagccatggtaaaatgtctctgggtgcttctttagttggcactggcatctgctgtgtcctgctcctttacacgattctctgtcctggggaatgattggctctcttacaaaatggagcattcttctcaacttgccttccggtctcctttccagttcccacgacggcagccagcacccctgacgcagtcgacaagtacctggagacccccggagatgagaacgagcacgcccatttccagaaagccaaagagaggttggaagccaagcaccgagagagaatgtcccaggtacggagaaggcttccaacttctgctgttctgttgtctagggagatgcacgctcgcctctgcctcagacgggtagatacaaagtttaatttaaatgttttccacgaggacacagattgtagggttcccctacatctatccagtgtgcgatcacatcaaggaaaggcaagtacaagaggattttgaagcacataatcaattgtgcctctccgctaaagaaaaggtactctgcgagatggtgaggcaagggctgttaactgatacattgtagtaaactttgtgtgtgt

SEQ ID NO: 2 - DNA, 700 bp, Rattus rattus; identical to SEQ ID NO: 1

SEQ ID NO: 3 - DNA, 299 bp, Rattus rattus
aagagcactgtaggtcctgacccagccttaaacttactactctacacaggatgcctggccgagggagagctggaggtgacagatcagctcccagggcaaagcgaccaaccctgggagcaggccctgaaccgcttctgggattacctgcgctgggtgcagacgctttctgaccaggtccaggaagagctgcagagctcccaagtcacacaggaactgacgtgagtgctcagcgcttcaccctccgcacctgctgagtatccagatccaggggttcctcctatctgggcacctacctactt

SEQ ID NO: 4 - DNA, 400 bp, Rattus rattus
caagagcactgtaggtcctgacccagccttaaacttactactctacacaggatgcctggccgagggagagctggaggtgacagatcagctcccagggcaaagcgaccaaccctgggagcaggccctgaaccgcttctgcgattacctgcgctgggtgcagacgctttctgaccaggtccaggaagagctgcagagctcccaagtcacacaggaactgacgtgagtgctcagcgcttcaccctccgcacctgctgagtatccagatccaggggttcctcctatctgggcacctacctacttgtttcctttctccatgagtgtgtgggccaggttggccttgaactctcaatacttctgctttctttagccttctggatactgggatgaacaggcattcatt

SEQ ID NO: 5 - DNA, 400 bp, Rattus rattus
tcgaagagctgctggatgaggaccagaaggttcggcccaacgaagaaaaccataaggacgcggacttgtacacttcccgggtgatgctcagcagtcaagtgcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcataga

SEQ ID NO: 6 - DNA, 300 bp, Rattus rattus
gcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcataga

SEQ ID NO: 7 - DNA, 19 bp, Rattus rattus
gccagcacccctgacgcag

SEQ ID NO: 8 - DNA, 17 bp, Rattus rattus
tcgacaagtacctggag

SEQ ID NO: 9 - DNA, 28 bp, Rattus rattus
aagcggttcagggcctgctcccagggtt

SEQ ID NO: 10 - DNA, 28 bp, Rattus rattus
gggattacctgcgctgggtgcagacgct

SEQ ID NO: 11 - DNA, 28 bp, Rattus rattus
cggggtcggagtggcgccgaaccctcat

SEQ ID NO: 12 - DNA, 28 bp, Rattus rattus
cggggtcggagtggcgccgaaccctcat

SEQ ID NO: 13 - protein, 35 aa, Artificial Sequence
  SYNTHESIZED
RSDALSVDSSHRTRRSDNLSETSGSLTRRSDDLTR

SEQ ID NO: 14 - protein, 35 aa, Artificial Sequence
  SYNTHESIZED
RSDHLSRQSSDLRRRSDVLSADRSNRIKTSSNLSR

SEQ ID NO: 15 - protein, 35 aa, Artificial Sequence
  SYNTHESIZED
DRSDLSRDRSHLARRSHNLARRSDDLSKRSAHLSR

SEQ ID NO: 16 - protein, 35 aa, Artificial Sequence
  SYNTHESIZED
RSDNLARQSSDLRRRSSHLSRRSDALSRDRSDLSR